(19) United States
(12) Patent Application Publication
(10) Pub. No.: US 2007/0104677 A1
(43) Pub. Date: May 10, 2007

(54) Preparation in the form of an emulsion

(76) Inventor: Barbara Dold, Heroldsbach (DE)

Correspondence Address: BACHMAN & LAPOINTE, P.C., 900 CHAPEL STREET, SUITE 1201, NEW HAVEN, CT, 06510, US

(21) Appl. No.: 10/551,863

(22) PCT Filed: Mar. 30, 2005
(86) PCT No.: PCT/EP05/03329

§ 371 Date: Sep. 30, 2005

(30) Foreign Application Priority Data
Apr. 2, 2004 (EP) 04008126.7

(51) Int. Cl. A61K 31/74

(52) U.S. Cl. 424/78.02

(57) ABSTRACT

A preparation for application skin and mucous membrane comprises an emulsion having an aqueous phase which contains a film-forming polymer combination and an emulsified phase having at least one pigment.

### BACKGROUND OF THE INVENTION

The invention concerns a cosmetic preparation in the form of an emulsion which is suitable for application to skin and mucous membrane, in particular in the region of the face and neck.

Cosmetic products which are used to improve appearance and which are applied skin and/or mucous membrane, in particular in the region of face and neck are known in many different forms. Those compositions usually incorporate substances which are intended to impart advantageous properties to the skin or which are intended to improve appearance. Thus it is known to incorporate into skin care compositions particulate materials which conceal fine lines, wrinkles, pores and other unwanted forms of the skin surface, by suitably reflecting light. That is described for example in “Quantification of the Soft-Focus Effect”, Cosmetics and Toiletries, Volume 111, July 1996, pages 57 through 61, with reference to substances such as TiO₂. In addition U.S. Pat. No. 4,892,726 discloses polymethylsilsesquioxane powders for incorporation into makeup and cosmetic compositions which, after application, impart to the skin a smooth appearance and a natural color. Corresponding products can be obtained under the brand “tospearl®”. U.S. Pat. No. 5,223,559 describes the use of small particles of a size in the range of between 0.5 and 50 μm for concealing skin defects. In addition EP-A 692 242 discloses the use of hollow particles of a size in the range of between 1 and 250 μm, for the same purpose. Other particles such as nylon particles are also already used in cosmetic preparations.

EP-A 1 156 772 discloses topical compositions which are not intended for the face but for application to other parts of the body, in particular the hands, which contain a particulate material in combination with special pigments and matting agents in a dermatologically acceptable topical carrier.

The known cosmetic compositions for topical application are all based on wax-bearing, fat-bearing and/or oil-bearing base substances which have proven to be disadvantageous. When a composition of that kind is applied to the skin of the face, the skin cannot absorb the fat and/or oil or cannot completely absorb same, so that a fine film of fat remains on the surface. The pigments contained in the composition float in that fat component which can move on the skin and which has a tendency to move into depressions such as wrinkles and small folds. That involves an accumulation of the pigments in small folds, pores, recesses and similar irregularities, which emphasizes them. That is undesirable as it is precisely pores, wrinkles and irregularities that are to be concealed.

A further problem is that the preparation when applied to a region either moves away from the point of application, which gives rise to a non-esthetic appearance, or however it clings to and discolors articles or skin, for example of the hand, which come into contact therewith. Both are unwanted.

In addition EP-A 0 793 957 discloses a cosmetic composition containing a film-forming polymer in aqueous dispersion. Pigments can be added to that composition. A colored layer can be applied to the skin by means of such a dispersion which is intended in particular for use as lipstick. It will be noted however that a fairly long period of time is required until the film has dried and thus no longer feels sticky or oily, but is light and scarcely perceptible.

Now, the object of the invention was to provide a cosmetic preparation which can be applied to the skin or mucous membrane, which by virtue of its composition conceals or hides folds, which remains clinging to the point of application and does not move away, and also does not transfer onto articles with which the composition comes into contact. In addition the invention seeks to provide a product which very rapidly achieves the desired effect after being applied and which does not feel sticky.

### SUMMARY OF THE INVENTION

The object is attained by a preparation in the form of an emulsion for application to skin and mucous membrane, which has an aqueous phase with a film-forming polymer combination comprising at least two polymers and an emulsified phase with at least one volatile silicone, at least one pigment and possibly fillers.

### DETAILED DESCRIPTION

Surprisingly it was found that, with the preparation according to the invention, it is possible to produce on the skin or mucous membrane a long-lasting film which gives the lady wearer a very pleasant feel, which clings to the point of application without moving away therefrom, but which on the other hand is so elastic that it complies with all movements of the skin or mucous membrane, without detaching. In addition the polymers of the composition on the one hand form a film and on the other hand, in co-operation with the pigments and possibly the fillers, conceal irregularities, wrinkles and pores in the skin. All those properties can be achieved without natural fats and oils or waxes being contained in the composition.

The preparation according to the invention is in the form of a composition of a consistency which can extend from being fluid to paste-like. It is therefore both suitable for fluid make-ups and also for creamy products such as concealers, camouflage creams or theater make-up. The preparation can be applied to skin, mucous membrane or also semi-mucous membrane and is intended in particular for application to face and neck, for example the skin of the face, the region around the eyes, the lips and the area around the lips and so forth. Application to décolletage, upper arms and other regions of the body where a concealing effect is to be achieved is also a possibility.

The preparation according to the invention is essentially based on film-forming substances, coloring agents and volatile substances, namely water and at least one volatile silicone.

When hereinafter reference is made to percentages for constituents, they are always to be interpreted as percent by weight with respect to the total weight of the preparation, unless otherwise stated.

An important ingredient of the preparation according to the invention is a film-forming polymer combination comprising at least two polymers. The term “film-forming polymer combination comprising at least two polymers” is used in the context of the present invention to denote a combination of two polymers which can either be in the form of a copolymer, preferably a block copolymer, with components made up of two different monomers, or however a polymer blend which is formed from at least two different polymers. What is essential is that the combination can form on the skin a film which is elastic and nonetheless firm or tenacious.

It has been found that it is necessary to use two different kinds of polymer to achieve a film having the desired properties. One polymer contributes to the elasticity and the other provides the strength of the film. In that respect in accordance with the invention it is possible both to use a copolymer made up of two different monomers, preferably in the form of blocks, for example as A-B, A-B-A, or A-B-A-B-A etc., and also a polymer blend comprising at least two different polymers. The important point is that the film obtained is flexible but strong and substantially non-movable.

Preferably therefore a film-forming polymer combination comprising an elastic polymer and a polymer imparting strength or toughness is used for the preparation according to the invention. Preferably polymers or copolymers of acrylate or derivatives thereof, methacrylate or derivatives thereof, polyvinylalcohol and/or polyvinylacetate or copolymers comprising two or more of the stated monomers or mixtures thereof are used as the elastic polymer. Particularly preferably polyurethanes, polyetherurethanes, polyesterurethanes, polyvinylpyrrolidone or mixtures thereof are used as the polymer imparting strength or toughness.

It was found that this combination of properties is achieved when either a mixture of polyurethane polymer and acrylate or methacrylate polymer or copolymer is used, or however a polyurethane acrylate copolymer. When a polymer is identified as “acrylate” here and in the claims, that is intended to denote polymers which were polymerized or copolymerized from acrylic acid or derivatives thereof. Thus the expression “acrylate” also includes acrylamides, acrylic esters such as acrylic acetates etc. but also copolymers in which the acrylate is the predominantly present polymer. The expression “polyurethane” generally embraces polymers which contain reaction products of bifunctional and polyfunctional isocyanates with hydroxyl group-bearing monomers such as polyhydric alcohols, or polyesters and polyethers.

In particular those polymers or copolymers which are derived from acrylic acid or methacrylic acid and derivatives thereof such as amides and esters, in particular acrylic acid, acrylamides and acrylic esters, fall to be considered for the film-forming polymer combination according to the invention. They can optionally have basic comonomers such as primary, secondary, tertiary or quaternary amino substituents, for example quaternisation products of dimethylaminoethylmethacrylate or diallyldimethylammoniumacrylic acid components; hydrophobic substituents such as long-chain alkyl groups with between 10 and 30 and preferably between 12 and 24 C-atoms, acid substituents such as sulfonate, phosphate and carboxyl groups or hydroxyl group-bearing components such as vinyl alcohol. Copolymers of vinylpyrrolidone/acrylate or dialkylaminoalkylmethacrylate are also suitable. The polyurethanes also include polyurethane polyvinylpyrrolidones, polyester polyurethanes, polyether polyurethanes and polyureas. The polyurethane can be for example an aliphatic, cycloaliphatic or aromatic polyurethane, polyurea/urethane or polyurea copolymer. Polyurethane can also be produced from polyesters which are straight-chain or branched, or alkyd compounds which have labile hydrogen. They can also have acid or basic groups.

Many cosmetically acceptable polyurethanes and polyurethane copolymers are known, which form the desired films and which occur in aqueous dispersion or solution.

Acrylic polymers can be produced by polymerization or copolymerization of acrylic acid, methacrylic acid, acrylic acid ester, methacrylic acid ester, acrylamide and/or methacrylamide monomers. Examples of suitable monomers that may be mentioned are methylmethacrylate, Ethylmethacrylate, butylmethacrylate, isobutylmethacrylate, 2-ethylhexylmethacrylate and laurylmethacrylate, N-tert.-butylacrylamide, N-tert.-octylacrylamide and hydroxyalkylacrylates. They can optionally be copolymerized in combination with vinyl monomers such as vinyl esters, vinyl acetate, vinyl alcohol, styrene or butadiene. Polymers and copolymers of vinylpyrrolidone, optionally in combination with acrylate or methacrylate, polyvinylalcohols and polyvinylacetates are also polymers which are suitable for the invention. Vinylpyrrolidone-based polymers or polyurethane-like polymers contribute to the strength of the film formed while the acrylates, methacrylates, polyvinylalcohols and polyvinylacetates afford the elastic properties.

In order to influence further properties of the film, further monomers can also be copolymerized or incorporated as blocks into the copolymers, or further polymers can be added to the polymer blends. Thus it is advantageous also to incorporate a polymer which imparts hydrophobic properties. Fatty acid ethers and esters are considered here. Alkyl sebacates and stearyl ethers and esters have proven to be particularly suitable.

Particularly resistant, elastic films can be obtained with the hereinafter listed polymers and copolymers and in particular a combination of at least two of the stated polymers or copolymers (specified in each case with INCI names): octadecene/MA copolymer/diethylhexyl-sebacates, acrylates copolymer, polyurethane-acrylates copolymer, lauryl dimethicone, PVP/dimethyl aminoethylmethacrylate/polycarbamyl/polyglycolester, PVP/dimethiconylacrylate/polycarbamyl/polyglycolester, PPG/IPDI/DPMA crosspolymer, alkylacrylate copolymer, acrylates/stearyl acrylate/dimethicone methacrylate copolymer, polyvinyl stearyl ether, acrylates/lauryl acrylate/stearyl acrylate/ethylamine oxide methacrylate copolymer.

The proportion of the respectively employed polymers is so selected that the desired properties are achieved. That can be established by a few routine tests. Suitable substances are for example those polymer blends or copolymers which form films which at body temperature exhibit elongation at tear of 150% or more, preferably 200% or more. The elongation at tear can be determined in accordance with the standard ASTM-D2370-92.

The polymers and copolymers or polymer blends are preferably used in the form of a solution and/or dispersion on a water base. Such solutions and dispersions are commercially available. The solids content of those solutions or dispersions is usually in a range of between 20 and 60%, preferably between 30 and 50%.

The film-forming polymer combination is contained in the preparation according to the invention in such a proportion that a sufficiently stable film can be formed after application to the skin or mucous membrane. For that purpose, the polymer solution or dispersion (that is to say the aqueous solution or dispersion containing the film-forming polymer combination) is generally added in a proportion of between 5 and 50% by weight with respect to the weight of the preparation. Preferably between 8 and 35% by weight, more preferably between 5 and 25% by weight of polymer solution or dispersion is used, particularly preferably between 8 and 18% by weight.

The film formed from the film-forming polymer combination according to the invention fixes the further ingredients and holds them firmly on the skin until it is detached again.

The aqueous phase of the preparation according to the invention is formed from the film-forming polymer combination as defined hereinbefore and optionally a further proportion of an aqueous medium, preferably water. As stated hereinbefore, polymer solutions and dispersions are commercially available, which then when used in the preparation according to the invention can also be “diluted” with water if that seems necessary. Therefore, depending on the desired viscosity and the desired water content and depending on the water content of the polymer solution or polymer dispersion used, aqueous medium, in particular water can also be added to the preparation according to the invention, in which respect the proportion of water is not critical and is determined only by the desired properties. In general the additional proportion of water, that is to say the proportion of water which is added in addition to the proportion of water contained in the polymer dispersion or polymer solution, is in a range of between 0 and 60%, preferably between 20 and 50%.

A further essential constituent of the preparation according to the invention is the proportion of coloring and/or colorless pigments, which provides for the esthetic effect. The term “pigment” is used in the context of the present invention not only to denote the conventional inorganic pigments but generally colorless, white or colored inorganic and organic pigments, but also color lacquers, pearlescent agents and so-called light diffusing pigments or “LDP” and lakes of organic dyes. In addition metal powders and white or colorless powders such as mica or kaolin are to be included among that constituent.

At least one pigment must be contained in the preparation according to the invention, preferably being an LDP or a light-reflecting pigment. Further pigments for particular effects or for coloration purposes can be added. By way of example mention may be made as inorganic pigments for example of yellow, black or red iron oxides, ultramarine, chromium oxide green, chromium hydroxide green, carbon black; organic pigments, lakes of organic dyes, carmine, flake-form metal powder such as passivated aluminum, brass, bronze, copper, silver or gold; mica, mica coated with metal oxides, for example with titanium dioxide, iron oxides, chromium oxide or chromium hydroxide; flake-form preparations based on silicon dioxide, aluminum oxide or glass, which can possibly also be coated with metal oxides, titanium dioxide, iron oxide, chromium oxide, or chromium hydroxide, and mixtures thereof.

The pigments are usually employed in very finely divided form so that they distribute well and achieve a uniform effect. Possibly the pigments can be so coated or derivatized that they are particularly well compatible with the other constituents. Methods of working pigments and derivatization thereof are known to the man skilled in the art.

Depending on the desired effect and depending on the respectively desired degree of coloring, the pigments are used in a proportion of between 0.1 and 25% by weight, preferably between 0.1 and 15% by weight, with respect to the total composition. If a stronger coloration effect is wanted, it is also possible to use a higher proportion of pigment. In that case, care is to be taken to ensure that adequate stabilization is guaranteed.

In accordance with the invention both preparations which are only intended for concealing wrinkles and which therefore do not contain any colored pigments but only LDP, and also make-up products which have their own color shade and form a colored film on the skin, are considered. The preparation according to the invention can also be used in the form of camouflage products or also theater make-up, in which cases the proportion of pigments is rather in the upper range. If the preparation according to the invention is only to be used for making wrinkles around the eyes or the mouth “invisible”, the proportion of pigment is selected to be in the lower range.

In order for the pigments to be stably distributed in the preparation according to the invention and maintained dispersed, a carrier is necessary. That carrier is a further important ingredient of the preparation according to the invention and is a volatile silicone which serves as a “carrier” for the pigment and optionally further constituents. The term “volatile silicone” is used to denote silicones of rather low molecular weight which evaporate relatively quickly at the temperature of the skin. In particular consideration is given to those silicones whose flash point is below 50° C., preferably below 40° C., with a corresponding evaporation kinetic. Volatile silicones are frequently used for cosmetic products and the cosmetically acceptable products are suitable for the present invention.

Silicones of that kind are well-known to the man skilled in the art. Examples that may be mentioned are cyclic dimethicone or cyclomethicone with between 3 and 7 Si-units, linear dimethylsiloxanes, dimethicone or methicone or derivatives thereof, for example those with up to 100, preferably between 2 and 20 siloxane units, which at the ends and/or in the chain can have alkyl residues with between 1 and 6 C-atoms and/or phenyl residues, and in particular dimethicone, hexamethyldisiloxane, cyclomethicone, dodecamethylcyclohexasiloxane, decamethylacyclopentasiloxane, octamethylcyclotetrasiloxane, and derivatives thereof as well as mixtures of the specified constituents. Further volatile compounds can possibly be added, which are compatible with the silicones, for example ethyl perfluoroisobutyl ether or ethyl perfluorobutyl ether.

The preparation according to the invention can also contain fillers which serve to move into the skin depressions, wrinkles, pores etc. to “fill them up” and thereby produce a smoother skin image. Those fillers can also afford a pleasant feel upon being applied and after application to the skin, at the location of application. Preferably inorganic or organic fine powders are used as the fillers. Examples are dimethicone/vinyldimethicone crosspolymer, polyamides, polyethylene, kaolin, talcum, boron nitride, PMMA, methylmethacrylate crosspolymer, silica, alumina, mica, PTFE, lauroyllysin and others. A mixture of those substances can also be used if required.

The proportion of the fillers is so adjusted that the dynamic viscosity of the preparation is in a suitable range and the desired effect is achieved. Proportions in the range of between 0.5 and 10% by weight, preferably between 1.5 and 8% by weight, have proven to be suitable.

The preparation according to the invention is in the form of an emulsion, wherein fillers and pigments dispersed in silicone form the emulsified phase and an aqueous solution or dispersion of film-forming polymers, possibly with added aqueous medium, forms the continuous phase. In order to produce the emulsion and keep it stable, one or more emulsifiers can be used. In that respect the preference is to use non-ionogenic emulsifiers in order to avoid reactions such as to change the properties, as between the emulsifier and cationic or anionic constituents. A further advantage of the present invention is that it is possible to dispense with the use of polyethylene glycol-bearing or polypropylene glycol-bearing emulsifiers which can have unpleasant side effects. Emulsifiers based on polyhydroxy compounds, sugar and sugar derivatives or fatty acids have proven to be suitable for the preparations according to the invention. Particularly suitable are polyglyceryl-3-methylglucose distearate, perfluorononyl dimethicone and derivatives, alkyl dimethicone, acrylates/stearyl acrylate/dimethicone methacrylate copolymer, hydroxypropyl dimethicone behenate, dimethicone propyl PG-betaine, polyglyceryl-4-isostearate, glyceryl stearate, glyceryl stearate citrate and caprylyl dimethicone ethoxyglucoside.

If used, the emulsifiers are preferably added in a proportion of between 3 and 10% with respect to the total composition.

As stated above the emulsion according to the invention can be of a consistency from being capable of flow to pasty. In order to set the appropriate viscosity for the respective form, a thickener can be added to the composition. All cosmetically acceptable substances can be envisaged as the thickener, which have a thickening effect in the presence of water, are compatible with the film-forming polymers and other ingredients and afford stable products. In general, natural or synthetic polymers are used for that purpose. Examples are hydrocolloids such as xanthan gum, gum arabic, carob bean flour; gelatin; polymer thickeners such as for example polyacrylates and the products sold under the brand “carbopol®”, and so forth. The proportion of thickener is dependent on the desired viscosity and is usually in a range of between 0 and 5% by weight.

The dynamic viscosity of the emulsion according to the invention is dependent on the desired consistency but is generally in a range of between 3 and 50 Pa·s, preferably between 10 and 25 Pa·s, measured with a Brookfield viskosimeter with spindle 4 at 60 rpm⁻¹.

A further optional constituent which is intended to provide for a film which is pleasant on the skin is a moistening agent which can be used as required. Moistening agents are hygroscopic compounds, generally hydroxyl group-bearing compounds. Examples that may be mentioned can be glycols such as 1,2-propylene glycol, dipropylene glycol, tripolylene glycol, glycerin, diglycerin, triglycerin, butylene glycol, hexylene glycol; sugar and sugar alcohols such as mannitol, sorbitol, xylitol, glucose, fructose, saccharose, maltose; urea, lactic acid and its salts, for example sodium lactate, pyrrolidone carboxylic acid, its sodium salt and mixtures thereof. If used the moistening agent is generally employed in a proportion of between 0.1 and 5% by weight with respect to the total composition.

The pH-value of the emulsion according to the invention should be substantially in the neutral range in order not to irritate the skin. Usually, cosmetic products are of a pH-value in the range of between 5 and 8.5, preferably between 5.5 and 8.0, particularly preferably between 7.2 and 8.0, which corresponds to the tear fluid. Optionally the appropriate pH-value can be set in the usual manner by the addition of acid or basic substances, as are generally conventional in the cosmetics field.

As the preparation according to the invention contains a high proportion of water, it may be advantageous to use a preserving agent in order to prevent microbial attack. Preserving agents which are suitable for cosmetics are well-known to the man skilled in the art and any suitable agent can be used. The proportion of the preserving agents is in the usual range and generally does not exceed 1% by weight with respect to the total composition.

Further constituents which can be added to the composition according to the invention to achieve certain effects are antioxidants, vegetable or animal extracts, perfume mixtures, light protection filters and the like. All those agents are well-known to the man skilled in the art and those generally used in cosmetics can be employed here in the amounts which are normally employed. The proportion thereof is in the usual range and generally does not exceed 5% by weight, preferably 1% by weight, with respect to the composition.

The preparation according to the invention has a very pleasant consistency and can be easily applied to the skin. After application the volatile components evaporate and leave an elastic pleasant film behind on the skin. The pigments and fillers contained therein and in part also the polymers used are deposited in recesses such as pores and wrinkles and fill them up as a result. The elastic film provides that the surface of the skin is tightened. In that way the skin is given an optically improved, smooth appearance. The film formed exhibits reduced migration and non-transfer properties. It is resistant to sebum and limitedly resistant to water. The film applied to the skin can however be easily removed again with warm water and possibly some rubbing.

In addition, if desired, it is also possible to incorporate into the preparation according to the invention active substances which are intended to impart to the skin soothing, inflammation-inhibiting and like properties. Water-soluble vegetable and animal active substances are particularly suitable for that purpose. Mention is to be made in this respect for example of bisabolol, pantothenol, aqueous extracts of calendula, camomile, yarrow, fennel, St. John's wort and other extracts which are pleasant on the skin.

A further subject of the present invention is a process for the production of the above-described preparation. For that purpose pigments and, if employed, fillers, are distributed or dispersed in a volatile silicone, preferably by homogenization. An aqueous phase is further provided, which optionally contains preserving agent, moistening agent and other water-soluble constituents, possibly with the exception of the film-forming polymer combination. If an emulsifier is used it can also be added to one of the two phases prior to emulsification. If the emulsifier is of a wax-like consistency and melts above ambient temperature the two phases are raised to the corresponding temperature at which the emulsifier melts.

If the emulsifier is active at ambient temperatures emulsification can be effected at ambient temperature. In an embodiment of the process according to the invention silicone phase and aqueous phase are mixed together and homogenized to form the emulsion. Thereafter the solution or dispersion of the film-forming polymer or polymers is added and after mixing the emulsion can be filled into a container. In another embodiment, if processing takes place at ambient temperature and the polymer is not adversely affected by the homogenization operation, the film-forming polymer can already be added to the aqueous phase prior to emulsification and emulsification can be effected thereafter.

The resulting emulsion is stable and, when filled into containers, can be stored over a prolonged period of time. The fillers and pigments dispersed in the volatile silicone, in accordance with the invention, are kept stable in the aqueous emulsion in the silicone droplets which form the emulsified phase, and do not settle.

The preparation according to the invention can be present in the form of an Si/W emulsion or a W/Si/W emulsion. The essential point is that the continuous phase is an aqueous phase in which the silicone phase is emulsified. By virtue of that structure and the proportion of film-forming polymer combination, it is possible to dispense with the normal use of fats, waxes and oils (in the form of triglycerides, liquid fatty acid esters or mineral oils), as are otherwise necessary.

As the combination according to the invention of film-forming agents forms a soft, pleasant but nonetheless stable film, use of waxes is not required and the problems that they entail are accordingly avoided. The emulsions according to the invention also do not contain fats and oils.

As the silicone serves as a carrier for pigments and dyes as well as fillers but the outer phase is formed by water and film-forming polymers dispersed or dissolved therein, a film which includes the pigments and fillers can be formed upon application so that, after evaporation of silicone and water, they are firmly anchored in the film and cannot migrate out of the film and into wrinkles.

The thickener can either be added to the aqueous phase prior to the emulsification operation, in particular if it has to swell prior to emulsification. It can however also be added together with the film-forming polymer after the emulsion is finished.

As stated above, on the one hand pigments contained in the emulsion and possibly fillers provide that wrinkles and recesses in the skin are filled up and optically concealed, and LDPs provide that recesses are optically lightened and the film formed from the film-forming agents causes tightening of the skin so that overall a very flat esthetic skin appearance is produced, without destroying the microrelief of the skin. At the same time the resulting film is resistant to perspiration and tears and does not transfer onto articles which come into contact therewith. As the preparation can also be colored as desired, it is excellently well suited as unpigmented linefiller and equally as pigmented make-up, as camouflage or as theater make-up, but also as wrinkle-concealing tightening foundation.

A further subject of the invention is therefore also the use of an emulsion having an aqueous phase which contains a film-forming polymer combination and an emulsified phase which contains one or more volatile silicones, at least one pigment and optionally fillers, for forming a wrinkle-concealing layer on skin and/or mucous membrane. In particular, the subject of the invention is the use of a preparation, as was described hereinbefore, for the production of a cosmetic composition having wrinkle concealing properties.

The preparation according to the invention is particularly suitable as make-up, foundation, concealer, eye cream or lip cream with a wrinkle-concealing effect. Preferred products are used to conceal wrinkles in the face, around the eyes, around the lips and/or on the neck.

The invention is described in greater detail by means of the Examples hereinafter.

The quantitative proportions are in % by weight with respect to the total weight of the preparation. The raw material names correspond to the “INCI names” (International Nomenclature of Cosmetic Ingredients) which are known to the man skilled in the relevant art.

### EXAMPLE 1

### Foundation for Face and Neck

| | | |
|---|---|---|
| A | Dimethicone/vinyldimethicone crosspolymer | 3.300 |
| | Dimethicone | 9.300 |
| | Decamethylcyclopentasiloxane | 12.300 |
| | Stearyl dimethicone | 3.800 |
| | Polyglyceryl-3-methylglucose distearate | 2.500 |
| B | Titanium dioxide (C.I.-No. 77.891) | 4.800 |
| | Yellow iron oxide (C.I.-No. 77.492) | 1.200 |
| | Red iron oxide (C.I.-No. 77.491) | 1.200 |
| | Black iron oxide (C.I.-No. 77.499) | 0.800 |
| | Silica and titanium dioxide and iron oxides | 1.700 |
| C | Aqua (distilled) | 40.300 |
| | Sorbitol | 4.500 |
| | Diazolidinyl urea | 0.200 |
| | Triethanolamine | 0.100 |
| D | Octadecene/MA copolymer and | 3.000 |
| | diethylhexyl sebacate* | |
| | Acrylates copolymer* | 2.500 |
| | Polyurethane-4* | 5.000 |
| | (* the quantitative proportions relate in each case to | |
| | the polymer dispersion) | |
| E | Bentonite | 3.500 |

The constituents of the phase A are introduced into a vessel and heated to 65° C. until a clear solution is produced, then introduced into a homogenization machine. The phase B is added and the mixture is mixed by means of a high-speed agitator until it is homogenous. In a separate vessel the phase C is raised to 65° C. and then sucked under slight vacuum into the mixture of phases A and B.

Thereafter the mixture is homogenized in a slight vacuum for 3-4 minutes and then cooled to about 40° C., then homogenization is effected for a further 2 minutes. The phase D is now added to the mixture, followed by intensive agitation for about 2 minutes, but it is no longer homogenized by means of the high-speed agitator, and the bentonite is now sprinkled into the mixture. Agitation is briefly effected with the agitator apparatus, then post-agitation is effected with the agitator apparatus for about 10 minutes and in that phase maximum vacuum is applied in order to deaerate the substance. Care is to be taken to ensure that the pH-value does not fall below 7.1, triethanolamine is possibly to be added. The pH-value of the finished preparation should be between 7.2 and 7.7 and should preferably be 7.5. The result obtained is a slightly light-brown preparation of a viscosity of between 5.5 and 7.5 Pa·s (neutral viscosity) which is particularly suitable for being filled into applicator devices as are described for example in U.S. Pat. No. 6,309,128 or U.S. Pat. No. 6,238,117. It will be appreciated that this preparation can also be filled into suitable containers in the usual manner.

### EXAMPLE 2

### Pasty Couperose Cream

| | | |
|---|---|---|
| A | Dimethicone/vinyldimethicone crosspolymer | 3.800 |
| | Dimethicone | 10.300 |
| | Decamethylcyclopentasiloxane | 12.300 |
| | Stearyl dimethicone | 3.800 |
| | Polyglyceryl-3-methylglucose distearate | 2.500 |
| B | Titanium dioxide (C.I.-No. 77.891) | 4.800 |
| | Yellow iron oxide (C.I.-No. 77.492) | 0.600 |
| | Chromium hydroxide (C.I.-No. 77.289) | 1.200 |
| | Titanium mica (C.I.-No. 77.891) | 2.500 |
| C | Aqua (distilled) | 38.400 |
| | Sorbitol | 3.500 |
| | Diazolidinyl urea | 0.200 |
| | Triethanolamine | 0.100 |
| D | Octadecene/MA copolymer and diethylhexyl | 3.000 |
| | sebacate* | |
| | Acrylates copolymer* | 2.500 |
| | Polyurethane-4* | 5.000 |
| | (* the quantitative proportions relate in each case to | |
| | the polymer dispersion) | |
| E | Bentonite | 4.500 |

Production is effected similarly to Example 1. The result obtained is a slightly light-green pasty preparation with good coverage of a viscosity of between 7.5 and 10.5 Pa·s which provides good coverage for skin reddenings and fine blood vessels and thus provides for color neutralization thereof. After drying a normal make-up can be applied over a skin which has been pre-treated in that way, with good success.

### EXAMPLE 3

### Pasty Concealer

| | | |
|---|---|---|
| A | Dimethicone/vinyldimethicone crosspolymer | 3.300 |
| | Dimethicone | 10.300 |
| | Decamethylcyclopentasiloxane | 12.300 |
| | Stearyl dimethicone | 3.800 |
| | Polyglyceryl-3-methylglucose distearate | 2.500 |
| B | Titanium dioxide (C.I.-No. 77.891) | 1.300 |
| | Yellow iron oxide (C.I.-No. 77.492) | 0.600 |
| | Red iron oxide (C.I.-No. 77.491) | 0.800 |
| | Black iron oxide (C.I.-No. 77.499) | 0.250 |
| | Mica (C.I.-No. 77.891) | 3.500 |
| | Silica and titanium dioxide and iron oxides | 2.300 |
| C | Aqua (distilled) | 39.250 |
| | Sorbitol | 3.500 |
| | Diazolidinyl urea | 0.200 |
| | Triethanolamine | 0.100 |
| D | Octadecene/MA copolymer and | 3.000 |
| | diethylhexyl sebacate* | |
| | Acrylates copolymer* | 3.500 |
| | Polyurethane-4* | 5.000 |
| | (* the quantitative proportions relate in each | |
| | case to the polymer dispersion) | |
| E | Bentonite | 4.500 |

Production is effected similarly to Example 1. The result obtained is a relatively transparent, almost skin-colored preparation with a slight sheen, which provides very good coverage of fine wrinkling of the skin, in particular in the region around and below the eyes. That preparation can remain as it is on the skin—however it can also serve as the basis for make-up after it has dried. It is of a viscosity in the range of between 7 and 12 Pa·s.

1-32. (canceled)

33. A preparation for application to skin and/or mucous membrane, comprising an emulsion having an aqueous phase comprising a film-forming polymer combination comprising at least two polymers and an emulsified phase comprising at least one volatile silicone, at least one pigment and optional fillers.

34. A preparation as set forth in claim 33, wherein in that the film-forming polymer combination has an elastic polymer and a polymer imparting strength or toughness.

35. A preparation as set forth in claim 34, wherein the elastic polymer is selected from the group consisting of an acrylate or a derivative thereof, a methacrylate or a derivative thereof, a polyvinylalcohol and/or a polyvinylacetate or a copolymer comprising two or more of said monomers and mixtures thereof.

36. A preparation as set forth in 34, wherein the polymer imparting strength or toughness is selected from the group consisting of polyurethane, polyetherurethane, polyesterurethane, polyvinylpyrrolidone, and mixtures thereof.

37. A preparation as set forth in 33, wherein the film-forming polymer combination is a polyurethane acrylate copolymer.

38. A preparation as set forth in claim 37, wherein the film-forming polymer combination is a polymer blend selected from the group consisting of a polyurethane polymer, polyvinylpyrrolidone polymer, acrylic polymer, acrylamide polymer, acrylic ester polymer, a polyvinylalcohol, polyvinylacetate and mixtures thereof.

39. A preparation as set forth in claim 33, wherein the polymer combination is selected from the group consisting of octadecene/MA copolymer/diethylhexyl-sebacates, acrylates copolymer, polyurethane-acrylates copolymer, lauryl dimethicone, PVP/dimethyl aminoethylmethacrylate/polycarbamyl/polyglycolester, PVP/dimethiconylacrylate/polycarbamyl/polyglycolester, PPG/IPDI/DPMA crosspolymer, alkylacrylate copolymer, acrylates/stearyl acrylate/dimethicone methacrylate copolymer, polyvinyl stearyl ether, acrylates/lauryl acrylate/stearyl acrylate/ethylamine oxide methacrylate copolymer, polyurethanes and mixtures thereof.

40. A preparation as set forth in 33, wherein the film-forming polymer is a combination of at least two polymers either in the form of a copolymer or in the form of a polymer blend.

41. A preparation as set forth in 33, wherein the film-forming polymer combination is a combination of one or more polyurethanes and one or more acrylates and/or methacrylates in the form of a copolymer or a polymer blend.

42. A preparation as set forth in 33, wherein the film-forming polymer selected from the group consisting of polymers, copolymers, crosspolymers of polyvinylpyrrolidone, polyvinylalcohol, polyvinylacetate, polyurethane, polyacrylate, polymethacrylate and mixtures thereof.

43. A preparation as set forth 33, further including at least one moistening agent contained in the aqueous phase.

44. A preparation as set forth in claim 43, wherein the moistening agent is selected from the group consisting of 1,2-propylene glycol, dipropylene glycol, tripolylene glycol, glycerin, diglycerin, triglycerin, butylene glycol, hexylene glycol; sugar, sugar alcohol, mannitol, sorbitol, xylitol; glucose, fructose, sucrose, maltose, urea, lactic acid in Na-lactate, sodium PCA and mixtures thereof.

45. A preparation as set forth in 33, wherein the at least one pigment comprises at least one spherical fine pigment in the form of LDP.

46. A preparation as set forth in 45, further including one or more pigments selected from the group consisting of organic pigments, inorganic pigments, lakes of organic dyes, metal powders, coated metal powders, flake-form preparations based on silicon dioxide, aluminum oxide, glass and mixtures thereof.

47. A preparation as set forth in claim 40, wherein the volatile silicone selected from the group consisting of dimethicone, hexamethyldisiloxane, cyclomethicone, dodecamethylcyclohexasiloxane, decamethylcyclopentasiloxane, octamethylcyclotetrasiloxane, ethyl perfluoroisobutyl ether, ethyl perfluorobutyl ether and mixtures thereof.

48. A preparation as set forth in 33, further including a filler.

49. A preparation as set forth 48, wherein the filler selected from the group consisting of dimethicone vinyldimethicone crosspolymers, polyamide, polyethylene, kaolin, talcum, boron nitride, PMMA, methylmethacrylate crosspolymer, silica, alumina, mica, PTFE, lauroyllysin and mixtures thereof.

50. A preparation as set forth in 33, further including an active substance.

51. A preparation as set forth in 33, wherein the preparation is free of wax, fat and oil.

52. A preparation as set forth in 33, further including an emulsifier.

53. A preparation as set forth in claim 52, wherein the emulsifier is a non-ionogenic emulsifier selected from the group consisting of polyglyceryl-3-methylglucose distearate, perfluorononyl dimethicone and derivatives thereof, alkyl dimethicone, acrylates/stearyl acrylate/dimethicone methacrylate copolymer, hydroxypropyl dimethicone behenate, dimethicone propyl PG-betaine, polyglyceryl-4-isostearate, glyceryl stearate, glyceryl stearate citrate, caprylyl dimethicone ethoxyglucoside and mixtures thereof.

54. A preparation as set forth in claim 33, further including additives and adjuvants which are usual in cosmetics.

55. A preparation as set forth in claim 54, wherein the additives selected from the group consisting of preserving agents, antioxidants, agents for regulating the pH-value, vegetable or animal extracts, perfume mixtures, light protection filters, and mixtures thereof.

56. A preparation as set forth in 33, wherein the film-forming polymer combination is used in the form of an aqueous solution.

57. A preparation as set forth in 33, wherein the film-forming polymer combination is used in the form of an aqueous dispersion.

58. A preparation as set forth in 33, wherein the film-forming polymer combination is contained in a proportion of between 5 and 50% by weight, with respect to the total preparation.

59. A preparation as set forth in 33, wherein the film-forming polymer combination is contained in a proportion of between 5 and 25% by weight, with respect to the total preparation.

60. A preparation as set forth in 33, wherein the at least one volatile silicone is contained in the preparation in a proportion of between 10 and 25% by weight.

61. A preparation as set forth in 33, wherein the at least one pigment is contained in the preparation in a proportion of between 0.1 and 15% by weight.

62. A process for the production of a preparation as defined in claim 33, wherein pigments and fillers are mixed with the volatile silicone, optionally preserving agent and moistening agent are incorporated in water, silicone phase and aqueous phase are homogenized to form an emulsion, optionally prior to or after the emulsification operation emulsifier is added and thickener and film-forming polymers are added and finally the substance is filled into containers.
